(19) United States
(12) Patent Application Publication
(10) Pub. No.: US 2013/0273010 A1
(43) Pub. Date: Oct. 17, 2013

(54) SERUM-FREE MEDIA AND THEIR USES FOR CHONDROCYTE EXPANSION

(71) Applicant: GENZYME CORPORATION, Cambridge, MA (US)

(72) Inventors: Stephen DUGUAY, Salem, MA (US); Barbara SEYMOUR, Natick, MA (US)

(73) Assignee: GENZYME CORPORATION, Cambridge, MA (US)

(21) Appl. No.: 13/864,149

(22) Filed: Apr. 16, 2013

Related U.S. Application Data
(63) Continuation of application No. 13/401,037, filed on Feb. 21, 2012, now abandoned.
Application No. 13/401,037 is a continuation of application No. 11/763,587, filed on Jun. 15, 2007, now abandoned.
(60) Provisional application No. 60/805,307, filed on Jun. 20, 2006.

(51) Int. Cl. C12N 5/071; A61K 35/32

(52) U.S. Cl. CPC C12N 5/0655; A61K 35/32
USPC 424/93.7; 435/405; 435/375; 435/325; 435/366

(57) ABSTRACT

The present invention provides defined serum-free cell culture media useful in culturing fibroblasts, especially articular chondrocytes, that avoid problems inherent in the use of serum-containing media. The defined media comprise platelet-derived growth factor (PDGF), chemically defined lipids, oncostatin M (OSM), interleukin-6 (IL-6), leukemia inhibitory factor (LIF), or combinations of these compounds. In another aspect, the present invention also provides tissue culture methods that comprise incubating chondrocytes in the defined serum-free media. The methods enhance attachment and proliferative expansion of chondrocytes seeded at low density while maintaining their redifferentiation potential.

This is a continuation of U.S. patent application Ser. No. 13/401,037, filed Feb. 21, 2012, which is continuation of U.S. patent application Ser. No. 11/763,587, filed Jun. 15, 2007, which claims the benefit of priority of U.S. Provisional Patent Application No. 60/805,307, filed on Jun. 20, 2006, all of which are incorporated herein by reference in their entirety.

### FIELD OF THE INVENTION

The present invention relates to the field of cell and tissue culture. More specifically, the invention relates to methods and compositions for ex vivo propagation of cells capable of forming cartilaginous tissue intended for treatment or repair of cartilage defects.

### BACKGROUND OF THE INVENTION

Articular cartilage is composed of chondrocytes encased within the complex extracellular matrix produced by those chondrocytes. The unique biochemical composition of this matrix provides for the smooth, nearly frictionless motion of articulating surfaces of the joints. With age, tensile properties of human articular cartilage change as a result of biochemical changes. After the third decade of life, the tensile strength of articular cartilage decreases markedly. Damage to cartilage produced by trauma or disease, e.g., rheumatoid and osteoarthritis, can lead to serious physical debilitation.

The inability of cartilage to repair itself has led to the development of several surgical strategies to alleviate clinical symptoms associated with cartilage damage. More than 500,000 arthroplastic procedures and joint replacements are performed annually in the United States alone. Autologous chondrocyte implantation is a procedure that has been approved for treatment of articular cartilage defects. The procedure involves harvesting a piece of cartilage from a non-weight bearing part of the femoral condyle and propagating the isolated chondrocytes ex vivo for subsequent implantation back into the same patient. Brittberg et al., New Engl. J. Med. 331:889-895 (1994).

Articular chondrocytes express articular cartilage-specific extracellular matrix components. Once articular chondrocytes are harvested and separated from the tissue by enzymatic digestion, they can be cultured in monolayers for proliferative expansion. However, during tissue culture, these cells adopt a fibroblastic morphology and cease to produce type II collagen and proteoglycans characteristic of hyaline-like articular cartilage. Such “dedifferentiated” cells proliferate rapidly and produce type I collagen, which is characteristic of fibrous tissue. Nevertheless, when placed in an appropriate environment such as suspension culture medium in vitro (Aulthouse et al., In Vitro Cell. & Devel. Biology 25:659-668 (1989)) or in the environment of a cartilage defect in vivo (Shortkroff et al., Biomaterials 17:147-154 (1996)), the cells redifferentiate, i.e., express articular cartilage-specific matrix molecules again. The reversibility of dedifferentiation is key to the successful repair of articular cartilage using cultured autologous chondrocytes.

Human chondrocytes are typically cultured in Dulbecco's Modified Eagle's Medium (DMEM) supplemented with 10% (v/v) fetal bovine serum (FBS). Aulthouse et al., In Vitro Cell. & Devel. Biology, 25:659-668 (1989); Bonaventure et al., Exp. Cell Res., 212:97-104 (1994). However, even though serum is widely used for mammalian cell culture, there are several problems associated with its use: (1) serum contains many unidentified or non-quantified components and therefore is not “defined;” (2) the composition of serum varies from lot to lot, making standardization difficult for experimentation or other uses of cell culture; (3) many of the serum components affect cell attachment, proliferation, and differentiation making it difficult to control these parameters; (4) some components of serum are inhibitory to the proliferation of specific cell types and to some degree may counteract its proliferative effect, resulting in sub-optimal growth; and (5) serum may contain viruses and other pathogens which may affect the outcome of experiments or provide a potential health hazard if the cultured cells are intended for implantation in humans. Freshney (1994) Serum-free media. In: Culture of Animal Cells, John Wiley & Sons, New York, 91-99.

Thus, the use of defined serum-free media is particularly advantageous in the ex vivo expansion of chondrocytes for treatment of cartilage defects. However, such defined serum-free media must be sufficient for attachment of adult human articular chondrocytes seeded at low density, sustain proliferation until confluent cultures are attained, and maintain the capacity of chondrocytes to re-express the articular cartilage phenotype.

There has been some effort to develop biochemically defined media (DM) for cell culture. DM generally includes nutrients, growth factors, hormones, attachment factors, and lipids. The precise composition must be tailored for the specific cell type for which the medium is designed. Successful growth of some cell types, including fibroblasts, keratinocytes, and epithelial cells, has been achieved in various DM. Freshney, 1994 and Butler M. et al., Appl. Microbiol. Biotechnol. 68:283-91 (2005).

The amounts of starting cell material available for autologous chondrocyte implantation are generally limited. Therefore, it is desirable to seed articular chondrocytes at a minimal subconfluent density. Attempts to culture articular chondrocytes at subconfluent densities in DM have been only partially successful. Although DM that can sustain the proliferative capacity of the chondrocytes seeded at low density have been developed, the use of these media still requires serum for the initial attachment of cells to the tissue culture vessel after seeding. Adolphe et al., Exp. Cell Res. 155:527-536 (1984), and U.S. Pat. No. 6,150,163.

Consequently, a need exists to optimize, standardize, and control conditions for attachment, proliferation and maintenance of redifferentiation-capable chondrocytes for use in medical applications, especially, in humans.

### SUMMARY OF THE INVENTION

This invention provides compositions of chemically defined culture media (DMs), methods of making such media, and methods of using such media, e.g., for culturing cells, in particular, human articular chondrocytes for repair of cartilage defects. One of the distinguishing features of the DM of the invention is the presence of one or more substantially pure cytokines of the IL-6 family, such as, e.g., oncostatin M (OSM), interleukin-6 (IL-6), and leukemia inhibitory factor (LIF).

Among other advantages, the invention allows one to avoid the use of serum in chondrocyte cultures, enhance cell attachment and proliferation under serum-free conditions, and/or to maintain the capacity of chondrocytes to re-express cartilage-specific phenotype. In one aspect, the invention provides DM that is sufficient for the initial attachment of cells to a culture substratum, thereby eliminating a need for a serum-containing medium in the initial stage of cell culture. Another aspect of the invention provides defined serum-free cell culture media that promote proliferation of cells such as chondrocytes without use of serum at any stage during cell culture. Yet another aspect of the invention provides cell culture media that may be used to prime chondrocytes prior to implantation into a subject or included as a redifferentiation-sustaining medium to chondrocytes embedded in a matrix intended for implantation into cartilage defects. Another aspect of the invention provides a method of culturing a chondrocyte to a state that is suitable for treating a patient suffering from a cartilage defect. Additional advantages of the invention will be set forth in part in the description which follows, and in part will be obvious from the description, or may be learned by practice of the invention.

The DM of the invention comprises a basal medium supplemented with one or more supplements, including one or more cytokines of the IL-6 family, such as, e.g., OSM, IL-6, and LIF.

The basal medium may be any suitable medium. In preferred embodiments, the basal medium is cDRF (Table 3) or cDRFm (Table 4). cDRF and cDRFm, are made by mixing DMEM, RPMI-1640, and Ham's F-12 at a 1:1:1 ratio or by appropriately combining pre-mixed media and adding certain growth supplements to arrive at the basal media as defined in Tables 3 and 4 respectively.

In further preferred embodiments, the basal medium is additionally supplemented with platelet-derived growth factor (PDGF) and/or one or more lipids. In some embodiments, the lipids are a chemically defined lipid mixture (CDLM; Table 5) or one or more lipids from CDLM (e.g., stearic acid, myristic acid, oleic acid, linoleic acid, palmitic acid, palmitoleic acid, arachidonic acid, linolenic acid, cholesterol, and alpha-tocopherol acetate). In some embodiments, a DM of the invention may include a basal medium (e.g., cDRF or cDRFm) supplemented with:
(a) one or both of substantially pure PDGF and CDLM; and
(b) one or more of substantially pure OSM, substantially pure IL-6, and substantially pure LIF.
   For example, in preferred embodiments, DM of the invention may include: (a) a basal medium; (b) 0.1-100 ng/ml PDGF; (c) 0.05-5% CDLM; (d) 0.01-10 ng/ml OSM; and/or (e) 0.01-10 ng/ml IL-6.

It is to be understood that both the foregoing general description and the following detailed description are exemplary and explanatory only and are not restrictive of the invention, as claimed.

### BRIEF DESCRIPTION OF THE DRAWINGS

FIG. 1 depicts a comparison in growth of primary human chondrocytes cultured in (1) DMEM+10% FBS or (2) the E93 medium (cDRFm, as defined in Table 4, supplemented with CDLM, PDGF, IL-6, and OSM) over three passages.

FIG. 2 depicts a comparison demonstrates a comparison in cell yield of primary human chondrocytes cultured in (1) DMEM+10% FBS or (2) the E93 medium (cDRFm, as defined in Table 4, supplemented with CDLM, PDGF, IL-6, and OSM) over three passages.

FIG. 3 shows an RPA of cell lysate from chondrocytes grown in E93 (lanes 2, 3, 4) or DMEM+10% FBS (lanes 5, 6, 7). The cartilage markers, collagen 2 and aggrecan, are expressed in all samples.

### DETAILED DESCRIPTION OF THE INVENTION

This invention provides compositions of chemically defined culture media (DMs), methods of making such media, and methods of using such media, e.g., for culturing cells, such as human articular chondrocytes, for repair of cartilage defects. The invention is based, at least in part, on the discovery that the basal medium referred to as cDRFm supplemented with PDGF and CDLM and one or more cytokines of the IL-6 family is sufficient for attachment, proliferation and maintenance of redifferentiation-capable chondrocytes in culture and can substitute for a serum-containing medium in all stages of cell culture. The cytokines of the IL-6 family of cytokines include, for example, OSM, IL-6, and LIF.

Accordingly, in one aspect, the invention provides a culture medium comprising a basal medium supplemented with one or more supplements, which include one or more cytokines of the IL-6 family, such as, e.g., OSM, IL-6, and LIF.

The term “supplemented with” indicates that a supplement has been added to a starting material to arrive at an ending material. Unless specifically indicated, the supplement or supplements need not be added at a specific time or in a specific order. The term “supplemented with” does not preclude the starting material from being additionally supplemented with other supplements, at any point in time, before or after being supplemented with the present supplement. Unless specifically indicated, supplements are added to the medium in a “substantially pure” form. The term “substantially pure” indicates that a supplement is substantially free of components with which it naturally occurs in nature. For example, a substantially pure cytokine could be a purified cytokine or a cytokine that is recombinantly produced.

### I. Preparation of Basal Medium

The first step in preparing defined, serum-free media (DM) of the invention is to obtain a basal medium. The basal medium may be any suitable medium. In illustrative embodiments, the basal medium is cDRF as defined in Table 3. cDRF can be prepared from commercially available starting components as described below. cDRF is a modification of the DM developed by Adolphe et al. (Exp. Cell Res. 155:527-536 (1984)) and by McPherson et al. (U.S. Pat. No. 6,150,163).

The three starting components of cDRF are DMEM, RPMI-1640, and Ham's F12 (Invitrogen; Carlsbad, Calif.). The starting components are combined at a 1:1:1 ratio. All three media can be combined at once, or any two of the media can be premixed and then combined with an appropriate amount of a third medium. The precise composition of starting components is set forth in Table 1. The resulting medium (defined in Table 2 and referred to as DRF) is then supplemented with ITS (10 μg/ml insulin, 5.5 μg/ml transferrin, 7 ng/ml selenium, and, optionally, 2.0 μg/ml ethanolamine; Invitrogen, Carlsbad, Calif.), human fibronectin (BD Biosciences; San Jose, Calif.), human serum albumin (HSA) (Grifols; Los Angeles, Calif.; or Baxter; Westlake Village, Calif.), linoleic acid (Sigma-Aldrich; St. Louis, Mo.), human basic fetal growth factor (bFGF) (R&D Systems, Minneapolis, Minn.), gentamycin (Invitrogen; Carlsbad, Calif.), and hydrocortisone (Sigma-Aldrich; St. Louis, Mo.) to create cDRF. All materials are reconstituted, diluted, and stored as per the supplier's recommendations. The exact order of combining components to arrive at a final medium is not essential. The complete medium may be prepared using standard laboratory techniques and stored preferably at 2-8° C. until use. In a preferred embodiment, the basal medium is prepared essentially as described above with adjustments to the amount of human serum albumin, linoleic acid, and hydrocortisone to arrive at modified cDRF (cDRFm) as defined in Table 4.

In some embodiments, the basal medium is a medium that comprises all essential components of cDRF listed in Table 3. A component or a subset of components listed in Table 3 is non-essential if, when its concentration is reduced, or the component is eliminated, the properties of the medium related to chondrocyte attachment, proliferation, and/or redifferentiation, remain substantially the same. The stated concentrations of individual components may be adjusted for specific cell culture conditions. Such adjustments can easily be made by a person skilled in the art using routine techniques.

Additional components may be added to the medium if such components are desirable and do not negatively impact on chondrocytes attachment, proliferation, and redifferentiation. Such components include, but are not limited to, growth factors, lipids, serum proteins, vitamins, minerals, and carbohydrates. For example, it may be advantageous to supplement the medium with growth factors or hormones that promote chondrocyte redifferentiation such as TGF-β (TGF-β1, -β2, -β3), IGF, and insulin, as described in U.S. Pat. No. 6,150,163. Such growth factors and hormones are commercially available. Additional examples of supplements include, but are not limited to, bone morphogeneteic proteins (BMPs), of which there are at least 15 structurally and functionally related proteins. BMPs have been shown to be involved in the growth, differentiation, chemotaxis, and apoptosis of various cell types. Recombinant BMP-4 and BMP-6, for example, can be purchased from R&D Systems (Minneapolis, Minn.). The concentration of various such supplements in DM of the invention can be determined with minimal experimentation. For example, the concentration of BMP in DM of the invention is chosen from 0.01-0.1 ng/ml, 0.1-1 ng/ml, 1-10 ng/ml, 100 ng/ml, 10-50 ng/ml, 50-100 ng/ml, and 0.1-1 μg/ml.

A skilled artisan will appreciate that DM of the invention have advantages in addition to avoiding the use of serum. However, it may be desirable to utilize the DM of the invention in applications where the use of undefined components is acceptable. Consequently, the DM of the invention may be supplemented with serum e.g., fetal calf serum, or other chemically undefined components such as, for example, animal or plant tissue extracts. Thus, in certain embodiments, the DM of the invention may be supplemented with 10% or less, for example, 8% or less, 6% or less, 4% or less, 2% or less, or 1% or less of serum.

A skilled artisan will also appreciate that equivalents of cDRF may be prepared from a variety of known media, e.g., Basal Medium Eagle medium (Eagle, Science, 122:501 (1955)), Minimum Essential medium (Dulbecco et al., Virology, 8:396 (1959)), Ham's medium (Ham, Exp. Cell Res. 29:515 (1963)), L-15 medium (Leibvitz, Amer. J. Hyg. 78:173 (1963)), McCoy 5A medium (McCoy et al., Proc. Exp. Biol. Med. 100:115 (1959)), RPMI medium (Moore et al., J. A. M. A. 199:519 (1967)), Williams' medium (Williams, Exp. Cell Res. 69:106-112 (1971)), NCTC 135 medium (Evans et al., Exp. Cell Res. 36:439 (1968)), Waymouth's medium MB752/1 (Waymouth, Nat. Cancer Inst. 22:1003 (1959)), etc. These media may be used singularly or as mixtures in suitable proportions to prepare a basal medium equivalent to cDRF. Alternatively, cDRF or its equivalent can be prepared from individual chemicals or from other media and growth supplements. The invention is not limited to media of any particular consistency and encompasses the use of media ranging from liquid to semi-solid and includes solidified media and solid compositions suitable for reconstitution.

TABLE 1
Compositions of Starting Medium

| | DMEM/F12 | RPMI-1640 |
|---|---|---|
| | 1x Liquid, mg/L | 1x Liquid, mg/L |
| Inorganic Salts: | | |
| CaCl₂ (anhyd.) | 116.6 | — |
| Ca(NO₃)₂•4H₂O | — | 100 |
| CuSO₄•5H₂O | 0.0013 | — |
| Fe(NO₃)₂•9H₂O | 0.05 | |
| FeSO₄•7H₂O | 0.417 | — |
| KCl | 311.8 | 400 |
| MgSO₄ (anhyd.) | 48.84 | 48.84 |
| MgCl₂ (anhyd.) | 28.64 | — |
| NaCl | 6995.5 | 6000 |
| NaHCO₃ | 1200 | 2000 |
| NaH₂PO₄•H₂O | 62.5 | — |
| Na₂HPO₄ (anhyd.) | 71.02 | 800 |
| ZnSO₄•7H₂O | 0.432 | — |
| Other Components: | | |
| D-Glucose | 3151 | 2000 |
| Glutathione | — | 1 |
| (reduced) | | |
| Hypoxanthine Na | 2.39 | — |
| Linoleic Acid | 0.42 | — |
| Lipoic Acid | 0.105 | — |
| Phenol Red | 8.1 | 5 |
| Putrescine 2HCl | 0.081 | 5 |
| Sodium Pyruvate | 55 | — |
| Thymidine | 0.365 | — |
| HEPES | — | 5300 |
| Amino Acids: | | |
| L-Alanine | 4.45 | — |
| L-Arginine | — | 200 |
| L-Arginine•HCl | 147.5 | — |
| L-Asparagine•H₂O | 7.5 | — |
| L-Asparagine (free base) | — | 50 |
| L-Aspartic Acid | 6.65 | 20 |
| L-Cystine•2HCl | 31.29 | 65 |
| L-Cysteine•HCl•H₂O | 17.56 | — |
| L-Glutamic Acid | 7.35 | 20 |
| L-Glutamine | 365 | 300 |
| Glycine | 18.75 | 10 |
| L-Histidine•HCl•H₂O | 31.48 | — |
| L-Histidine (free base) | — | 5 |
| L-Hydroxyproline | — | 20 |
| L-Isoleucine | 54.47 | 50 |
| L-Leucine | 59.05 | 50 |
| L-Lysine•HCl | 91.25 | 40 |
| L-Methionine | 17.24 | 15 |
| L-Phenylalanine | 35.48 | 15 |
| L-Proline | 17.25 | 20 |
| L-Serine | 26.25 | 30 |
| L-Threonine | 53.45 | 20 |
| L-Tryptophan | 9.02 | 5 |
| L-Tyrosine•2Na₂H₂O | 55.79 | 29 |
| L-Valine | 52.85 | 20 |
| Vitamins: | | |
| Biotin | 0.0035 | 0.2 |
| D-Ca pantothenate | 2.24 | 0.25 |
| Choline Chloride | 8.98 | 3 |
| Folic Acid | 2.65 | 1 |
| I-Inositol | 12.6 | 35 |
| Niacinamide | 2.02 | 1 |
| Para-aminobenzoic | — | 1 |
| Acid | | |
| Pyridoxine HCl | 2.031 | 1 |
| Pyridoxal HCl | — | — |
| Riboflavin | 0.219 | 0.2 |
| Thiamine HCl | 2.17 | 1 |
| Vitamin B₁₂ | 0.68 | 0.005 |

TABLE 2
Composition of DRF

| | 1x Liquid, mg/L |
|---|---|
| Inorganic Salts: | |
| CaCl₂ (anhyd.) | 77.7333 |
| Ca(NO₃)₂•4H₂O | 33.3333 |
| CuSO₄•5H₂O | 0.0009 |
| Fe(NO₃)₂•9H₂O | 0.0333 |
| FeSO₄•7H₂O | 0.2780 |
| KCl | 341.2000 |
| MgSO₄ (anhyd.) | 48.8400 |
| MgCl₂ (anhyd.) | 19.0933 |
| NaCl | 6663.6667 |
| NaHCO₃ | 1466.6667 |
| NaH₂PO₄•H₂O | 41.6667 |
| Na₂HPO₄ (anhyd.) | 314.0133 |
| ZnSO₄•7H₂O | 0.2880 |
| Other Components: | |
| D-Glucose | 2767.3333 |
| Glutathione (reduced) | 0.3333 |
| Hypoxanthine Na | 1.5933 |
| Linoleic Acid | 0.2800 |
| Lipoic Acid | 0.0700 |
| Phenol Red | 7.0667 |
| Putrescine 2HCl | 1.7207 |
| Sodium Pyruvate | 36.6667 |
| Thymidine | 0.2433 |
| HEPES | 1766.6667 |
| Amino Acids: | |
| L-Alanine | 2.9667 |
| L-Arginine | 66.6667 |
| L-Arginine•HCl | 98.3333 |
| L-Asparagine•H₂O | 5.0000 |
| L-Asparagine (free base) | 16.6667 |
| L-Aspartic Acid | 11.1000 |
| L-Cystine•2HCl | 42.5267 |
| L-Cysteine•HCl•H₂O | 11.7067 |
| L-Glutamic Acid | 11.5667 |
| L-Glutamine | 343.3333 |
| Glycine | 15.8333 |
| L-Histidine•HCl•H₂O | 20.9867 |
| L-Histidine (free base) | 1.6667 |
| L-Hydroxyproline | 6.6667 |
| L-Isoleucine | 52.9800 |
| L-Leucine | 56.0333 |
| L-Lysine•HCl | 74.1667 |
| L-Methionine | 16.4933 |
| L-Phenylalanine | 28.6533 |
| L-Proline | 18.1667 |
| L-Serine | 27.5000 |
| L-Threonine | 42.3000 |
| L-Tryptophan | 7.6800 |
| L-Tyrosine•2Na₂H₂O | 46.8600 |
| L-Valine | 41.9000 |
| Vitamins: | |
| Biotin | 0.0690 |
| D-Ca pantothenate | 1.5767 |
| Choline Chloride | 6.9867 |
| Folic Acid | 2.1000 |
| I-Inositol | 20.0667 |
| Niacinamide | 1.6800 |
| Para-aminobenzoic | 0.3333 |
| Acid | |
| Pyridoxine HCl | 1.6873 |
| Pyridoxal HCl | — |
| Riboflavin | 0.2127 |
| Thiamine HCl | 1.7800 |
| Vitamin B₁₂ | 0.4550 |

TABLE 3
Composition of cDRF

| | 1x Liquid |
|---|---|
| DRF (Table 2) | 99% |
| ITS-X supplement (insulin, | 1% |
| transferrin, selenium, | |
| ethanolamine) | |
| Supplements: | |
| Linoleic Acid | 5 μg/ml |
| Gentamycin | 50 μg/ml |
| Hydrocortisone | 40 ng/ml |
| Fibronectin | 1 μg/ml |
| Basic Fibroblast Growth Factor | 10 ng/ml |
| (bFGF) | |
| Human Serum Albumin | 1 mg/ml |

TABLE 4
Composition of cDRFm

| | 1x Liquid |
|---|---|
| DRF (Table 2) | 99% |
| ITS-X supplement (insulin, | 1% |
| transferrin, selenium, | |
| ethanolamine) | |
| Supplements: | |
| Gentamycin | 50 μg/ml |
| Hydrocortisone | 160 ng/ml |
| Fibronectin | 1 μg/ml |
| Basic Fibroblast Growth Factor | 10 ng/ml |
| (bFGF) | |
| Human Serum Albumin | 0.5 mg/ml |

### II. Supplementation of Basal Medium

A. Platelet-Derived Growth Factor (PDGF)

In some embodiments, the basal medium is supplemented with substantially pure PDGF.

PDGF is a major mitogenic factor present in serum but not in plasma. PDGF is a dimeric molecule consisting of two structurally related chains designated A and B. The dimeric isoforms PDGF-AA, AB and BB are differentially expressed in various cell types. In general, all PDGF isoforms are potent mitogens for connective tissue cells, including dermal fibroblasts, glial cells, arterial smooth muscle cells, and some epithelial and endothelial cell.

Recombinantly produced PDGF is commercially available from various sources. Human recombinant PDGF-BB (hrPDGF-BB) used in the Examples below was purchased from R&D Systems (Minneapolis, Minn.; catalog #220-BB) and reconstituted and handled according to the manufacturer's instructions. The E. coli expression of hrPDGF-BB and the DNA sequence encoding the 109-amino-acid-residue mature human PDGF-B chain protein (C-terminally processed from that ends with threonine residue 190 in the precursor sequence) is described by Johnson et al. (EMBO J. 3:921 (1984)). The disulfide-linked homodimeric rhPDGF-BB consists of two 109-amino-acid-residue B chains and has molecular weight of about 25 kDa. The activity of PDGF is measured by its ability to stimulate ³H-thymidine incorporation in quiescent NR6R-3T3 fibroblast as described by Raines et al. (Meth. Enzymol. 109:749-773 (1985)). The ED₅₀ for PDGF in this assay is typically 1-3 ng/ml.

The concentration of PDGF is chosen from 0.1-1 ng/ml, 1-5 ng/ml, 5-10 ng/ml, 10 ng/ml, 10-15 ng/ml, 15-50 ng/ml, and 50-100 ng/ml. In certain embodiments, cDRF is supplemented with 1-25 ng/ml, more preferably, 5-15 ng/ml and, most preferably, about 10 ng/ml of PDGF. In a particular embodiment, the PDGF is PDGF-BB. Alternatively, PDGF could be of another type, e.g., PDGF-AB, PDGF-BB, or a mix of any PDGF types. In related embodiments, the DM of the invention further or alternatively comprises additional supplements as described below.

B. Lipids

In some embodiments, the basal medium is supplemented with CDLM (Table 5) or, alternately, one or more lipids from CDLM.

Lipids are important as structural components as well as potential energy sources in living cells. In vitro, most cells can synthesize lipids from glucose and amino acids present in the culture medium. However, if extracellular lipid is available, lipid biosynthesis is inhibited and the cells utilize free fatty acids, lipid esters, and cholesterol in the medium. Serum is rich in lipids and has been the major source of extracellular lipid for cultured cells. Chemically undefined lipid preparations based on marine oils have been found to be effective in promoting growth of cells in serum free-media in several systems. See, e.g., Weiss et al., In Vitro 26:30A (1990); Gorfien et al., In Vitro 26:37A (1990); Fike et al., In Vitro 26:54A (1990). Thus, supplementation of serum-free media with various lipids to replace those normally supplied by serum may be desirable.

Suitable lipids for use in the DM of this invention include stearic acid, myristic acid, oleic acid, linoleic acid, palmitic acid, palmitoleic acid, arachidonic acid, linolenic acid, cholesterol, and alpha-tocopherol acetate. In one embodiment, the basal medium is supplemented with the chemically defined lipid mixture (CDLM), shown in Table 5. CDLM is available from Invitrogen. As supplied by Invitrogen, in addition to the lipid components, CDLM contains ethanol (100 g/L) and emulsifiers Pluronic F68® (100 g/L) and Tween 80® (2.2 g/L).

In practicing the methods of the invention, the concentrations of individual lipid components of CDLM shown in Table 5 may be adjusted for specific cell culture conditions. Such adjustments can easily be made by a person skilled in the art using routine techniques. Furthermore, not all components of CDLM may be essential. A component or a subset of components is non-essential if, when its concentration is reduced, or the component is eliminated, the properties of the medium related to chondrocyte attachment, proliferation, and redifferentiation, remain substantially the same.

In certain embodiments, the DM of the invention comprises at least one, two, four, six, eight, or all lipid components of CDLM. In one embodiment, the DM comprises PDGF and CDLM as defined in Table 5. In other nonlimiting embodiments, the DM comprises PDGF and lipid combinations as set forth in Table 6.

TABLE 5
Composition of chemically defined lipid mixture (CDLM)

| Lipid components | mg/L |
|---|---|
| DL-alpha-tocopherol acetate | 70 |
| Stearic acid | 10 |
| Myristic acid | 10 |
| Oleic acid | 10 |
| Linoleic acid | 10 |
| Palmitic acid | 10 |
| Palmitoleic acid | 10 |
| Arachidonic acid | 2 |
| Linolenic acid | 10 |
| Cholesterol | 220 |

TABLE 6
Illustrative Lipid Combinations

| No. | Lipid(s) |
|---|---|
| 1 | cholesterol |
| 2 | cholesterol, arachidonic acid |
| 3 | cholesterol, arachidonic acid, linoleic acid |
| 4 | cholesterol, arachidonic acid, linoleic acid, linolenic acid |
| 5 | cholesterol, arachidonic acid, linoleic acid, linolenic acid, alpha-tocopherol acetate |
| 6 | cholesterol, arachidonic acid, linoleic acid, linolenic acid, alpha-tocopherol acetate, stearic acid |
| 7 | cholesterol, arachidonic acid, linoleic acid, linolenic acid, alpha-tocopherol acetate, stearic acid |
| 8 | cholesterol, arachidonic acid, linoleic acid, linolenic acid, alpha-tocopherol acetate, stearic acid, |
| | myristic acid |
| 9 | cholesterol, arachidonic acid, linoleic acid, linolenic acid, alpha-tocopherol acetate, stearic acid, |
| | myristic acid, oleic acid |
| 10 | cholesterol, arachidonic acid, linoleic acid, linolenic acid, alpha-tocopherol acetate, stearic acid, |
| | myristic acid, oleic acid, palmitic acid |
| 11 | cholesterol, arachidonic acid, linoleic acid, linolenic acid, alpha-tocopherol acetate, stearic acid, |
| | myristic acid, oleic acid, palmitic acid, palmitoleic acid |
| 12 | arachidonic acid, linoleic acid, linolenic acid, alpha-tocopherol acetate, stearic acid, myristic |
| | acid, oleic acid, palmitic acid, palmitoleic acid |
| 13 | arachidonic acid, linoleic acid, linolenic acid, stearic acid, myristic acid, oleic acid, palmitic acid, |
| | palmitoleic acid |
| 14 | arachidonic acid, linoleic acid, linolenic acid, stearic acid, myristic acid, oleic acid, palmitic acid |
| 15 | arachidonic acid, linoleic acid, linolenic acid, stearic acid, myristic acid, oleic acid |
| 16 | arachidonic acid, linoleic acid, linolenic acid, stearic acid, myristic acid |
| 17 | arachidonic acid, linoleic acid, linolenic acid, acetate, stearic acid |
| 18 | arachidonic acid, linoleic acid, linolenic acid, stearic acid |
| 19 | arachidonic acid, linoleic acid, linolenic acid |
| 20 | arachidonic acid, linoleic acid |
| 21 | arachidonic acid |
| 22 | cholesterol, linoleic acid |
| 23 | cholesterol, linoleic acid, linolenic acid |
| 24 | cholesterol, linoleic acid, linolenic acid, stearic acid |
| 25 | cholesterol, linoleic acid, linolenic acid, stearic acid, myristic acid |
| 26 | cholesterol, linoleic acid, linolenic acid, stearic acid, myristic acid, oleic acid |
| 27 | cholesterol, linoleic acid, linolenic acid, stearic acid, myristic acid, oleic acid, palmitic acid |
| 28 | cholesterol, linoleic acid, linolenic acid, stearic acid, myristic acid, oleic acid, palmitic acid, |
| | palmitoleic acid |
| 29 | cholesterol, linoleic acid, linolenic acid, alpha-tocopherol acetate, stearic acid, myristic acid, |
| | oleic acid, palmitic acid, palmitoleic acid |
| 30 | linoleic acid |
| 31 | cholesterol, linoleic acid |
| 32 | cholesterol, arachidonic acid, linoleic acid |
| 33 | cholesterol, arachidonic acid, linoleic acid, linolenic acid |
| 34 | cholesterol, arachidonic acid, linoleic acid, linolenic acid, alpha-tocopherol acetate |
| 35 | cholesterol, arachidonic acid, linoleic acid, linolenic acid, alpha-tocopherol acetate, stearic acid |
| 36 | cholesterol, arachidonic acid, linoleic acid, linolenic acid, alpha-tocopherol acetate, stearic acid, |
| | myristic acid |
| 37 | cholesterol, arachidonic acid, linoleic acid, linolenic acid, alpha-tocopherol acetate, stearic acid, |
| | myristic acid, oleic acid |
| 38 | cholesterol, arachidonic acid, linoleic acid, linolenic acid, alpha-tocopherol acetate, stearic acid, |
| | myristic acid, oleic acid |
| 39 | cholesterol, arachidonic acid, linoleic acid, linolenic acid, alpha-tocopherol acetate, stearic acid, |
| | myristic acid, oleic acid, palmitic acid, palmitoleic acid |
| 40 | linolenic acid |
| 41 | cholesterol, linolenic acid |
| 42 | cholesterol, alpha-tocopherol acetate, stearic acid, myristic acid, oleic acid, palmitic acid, |
| | palmitoleic acid |
| 43 | cholesterol, alpha-tocopherol acetate |
| 44 | cholesterol, stearic acid, myristic acid, oleic acid, palmitic acid, palmitoleic acid |
| 45 | stearic acid, myristic acid, oleic acid, palmitic acid, palmitoleic acid |
| 46 | cholesterol, myristic acid, oleic acid, palmitic acid, palmitoleic acid |
| 47 | cholesterol, oleic acid, palmitic acid, palmitoleic acid |
| 48 | cholesterol, stearic acid, myristic acid, oleic acid, palmitic acid, palmitoleic acid |
| 49 | cholesterol, myristic acid, oleic acid, palmitic acid |
| 50 | cholesterol, arachidonic acid, linoleic acid, linolenic acid, palmitic acid, palmitoleic acid |

In certain embodiments, the concentration (v/v) of lipids in the culture medium is chosen from 0.05-0.1%, 0.1-0.5%, 0.5%, 0.5-1%, 1-2%, and 2-5%. In certain other embodiments, the DM is additionally supplemented with 1 to 25 ng/ml, more preferably, 5 to 15 ng/ml, and, most preferably, about 10 ng/ml of PDGF. In a particular embodiment, the DM comprises approximately 0.5% (v/v) CDLM and 10 ng/ml PDGF.

C. IL-6-Family Cytokines

Members of the IL-6 family of cytokines each can utilize a shared signal transducing receptor subunit, gp130, which is found in a wide range of cell types. See, e.g., Hirano et al. (2001) IL-6 Ligand and Receptor Family. In: Cytokine Reference, Academic Press, San Diego, 523-535. Examples of IL-6-family cytokines include, but are not limited to, oncostatin M (OSM), interleukin-6 (IL-6), leukemia inhibitory factor (LIF), ciliary neurotrophic factor (CNTF), interleukin-11 (IL-11), cardiotrophin 1 (CT-1), and neurotrophin 1/B cell-stimulating factor 3 (NNT-1/BSF-3). Cytokines of the IL-6 family have been found to regulate cell growth and differentiation in a wide variety of biological systems, including hematopoiesis, neurogenesis, and osteogenesis. Bruce et al., Prog. Growth Factor Res. 4:157-170 (1992).

1. Oncostatin M (OSM)

Human OSM is a secreted glycoprotein that is initially translated as a 252-amino-acid polypeptide with a 25-residue hydrophobic signal sequence at the N-terminus that is removed during the secretion process. An additional post-translational cleavage event removes 31 C-terminal residues, leaving a 192-amino-acid disulfide-linked mature protein. Rose et al., Proc. Nat. Acad. Sci. USA 88:8641-8645 (1991); Robinson et al., Cell 77:1101-1116 (1994). In humans, OSM binds and signals through two different receptor complexes—the LIF receptor (LIFR)/gp130 heterodimer and the OSM receptor (OSMR)/gp130 heterodimer. Binding to either receptor complex leads to activation of the Janus kinase/signal transducers and activators of transcription (JAK/STAT) and mitogen-activated protein kinase (MAPK) signaling pathways. Heinrich et al., Biochem. J. 374:1-20 (2003).

OSM has been reported to inhibit the growth of some, but not all, human tumor cell lines. In contrast, OSM has also been reported to stimulate the growth of some normal fibroblasts, such as human foreskin fibroblasts or WI-38 cells. Zarling et al., Proc. Nat. Acad. Sci. USA 83:9739-9743 (1986). Thus, OSM may be useful for stimulating the growth of certain cells in vitro. A more detailed description of OSM can be found in U.S. Pat. Nos. 5,202,116 and 5,814,307.

OSM is readily available from commercial sources. In the Examples below, a 196-amino-acid recombinant OSM produced in E. coli was obtained from R&D Systems (Minneapolis, Minn.) (catalog No. 295-OM, see also Linsley et al., Mol. Cell. Biol. 10:1882-1890 (1990)). The biological activity of OSM may be assayed by testing in a human erythroleukemic cell line proliferation assay, as described, e.g., in Kitamura et al., J. Cell Physiol. 140:323-334 (1989). In a preferred embodiment, human OSM, is used to produce the media of the invention. However, one skilled in the art would recognize that OSM from other species, naturally occurring mutants, and engineered mutants may also be effective.

2. Interleukin-6 (IL-6)

IL-6 has many alternative names, including: interferon β2; B-cell differentiation factor; B-cell stimulatory factor 2; hepatocyte stimulatory factor; hybridoma growth factor; and CTL differentiation factor. Human IL-6 is a 186-amino-acid secreted glycoprotein that is synthesized as a 212-amino-acid precursor protein. Matsuda et al., (2001) IL-6. In: Cytokine Reference, Academic Press, San Diego, 538-563. In humans, IL-6 binds and signals through a complex of the IL-6 receptor (IL-6R) and a gp130 homodimer. Binding of IL-6 to the IL-6R receptor leads to activation of the Janus kinase/signal transducers and activators of transcription (JAK/STAT) and mitogen-activated protein kinase (MAPK) signaling pathways. Heinrich et al., Biochem. J. 374:1-20 (2003).

IL-6 has been reported to induce differentiation of PC12 neuronal cells, to induce clonogenic maturation of bone marrow progenitor cells, and to induce the growth of T cells. In contrast, IL-6 has also been shown to inhibit the growth of myeloid leukemia cells and breast cancer cells. Thus, IL-6 may be useful for stimulating the growth of certain cells in vitro. A more detailed description of IL-6 biology can be found in U.S. Pat. No. 5,188,828.

IL-6 is available from commercial sources. In the Examples below, a 184-amino-acid recombinant IL-6 produced in E. coli was obtained from R&D Systems (Minneapolis, Minn.) (catalog No. 206-IL, see also Hirano et al., Nature 324:73-76 (1986)). The biological activity of IL-6 is assayed by testing in a plasmacytoma proliferation assay as described in, e.g., Nordan et al., J. Immunol. 139:813 (1987). In a preferred embodiment, human IL-6 is used to produce the media of the invention. However, one skilled in the art would recognize that IL-6 from other species, naturally occurring mutants, and engineered mutants may also be effective.

3. Leukemia Inhibitory Factor (LIF)

LIF has several alternative names, including: cholinergic differentiation factor; human interleukin in DA cells; differentiation stimulating factor; MLPLI; and Emfilermin. Human LIF is a 180-amino-acid secreted glycoprotein. Kondera-Anasz et al., Am. J. Reprod. Immunol. 52:97-105 (2004). In humans, LIF binds and signals through the LIF receptor (LIFR)/gp130 heterodimer. Binding of LIF to the LIF receptor leads to activation of the Janus kinase/signal transducers and activators of transcription (JAK/STAT) and mitogen-activated protein kinase (MAPK) signaling pathways. Heinrich et al., Biochem. J. 374:1-20 (2003).

LIF has been reported to inhibit the proliferation of M1 myeloid leukemia cells. See, e.g., U.S. Pat. No. 5,443,825. In contrast, LIF has also been reported to stimulate the growth of neurons as well as to promote the differentiation of neurons from an adrenal medullary phenotype to an acetylcholinergic phenotype. See, e.g., U.S. Pat. No. 5,968,905. The addition of LIF to severed nerves can also enhance nerve regeneration. See, e.g., U.S. Pat. No. 6,156,729. Thus, LIF may be useful for promoting the growth of certain cells in vitro.

LIF is available from commercial sources. In the Examples below, a 181-amino-acid recombinant human LIF produced in E. coli was obtained from Sigma-Aldrich (St. Louis, Mo.) (catalog No. L 5283, see also Gearing et al., EMBO J. 6:3995 (1987)). The biological activity of LIF is assayed by testing for its ability to stimulate the differentiation of M1 mouse myeloid leukemia cells as described, e.g., in Gearing et al., EMBO J. 6:3995 (1987). In a preferred embodiment, human LIF is used to produce the media of the invention. However, one skilled in the art would recognize that LIF from other species, naturally occurring mutants, and engineered mutants may also be effective.

In certain embodiments, the DM of the invention is cDRF supplemented with PDGF, one or more lipids selected from the group consisting of stearic acid, myristic acid, oleic acid, linoleic acid, palmitic acid, palmitoleic acid, arachidonic acid, linolenic acid, cholesterol, and alpha-tocopherol acetate, and one or more cytokines. In particular embodiments, DM of the invention is cDRF supplemented with PDGF, one or more lipids selected from the group consisting of stearic acid, myristic acid, oleic acid, linoleic acid, palmitic acid, palmitoleic acid, arachidonic acid, linolenic acid, cholesterol, and alpha-tocopherol acetate, and one or more of the group consisting of OSM, IL-6, and LIF. The concentration of cytokine is chosen from 0.01-0.1 ng/ml, 0.1-1 ng/ml, 1-5 ng/ml, 5-10 ng/ml, 10-15 ng/ml, 15-50 ng/ml, and 50-100 ng/ml. In certain embodiments, cDRF is supplemented with 0.01-10 ng/ml, more preferably, 0.1-2 ng/ml and, most preferably, 0.5-1 ng/ml of OSM, IL-6, and/or LIF. In a preferred embodiment, cDRF is supplemented with approximately 10 ng/ml PDGF, 0.5% CDLM, 1 ng/ml IL-6, and 0.5 ng/ml OSM. In related embodiments, the DM of the invention further comprises additional supplements as described below.

In certain embodiments, the DM of the invention comprises at least one, two, or all three of OSM, IL-6, and LIF. In other nonlimiting embodiments, the DM comprises combinations of OSM, IL-6, and LIF as set forth in Table 7. In additional nonlimiting embodiments, the DM comprises any combination of OSM, IL-6, and LIF set forth in Table 7, PDGF, and CDLM as defined in Table 5. In additional nonlimiting embodiments, the DM comprises any combination of OSM, IL-6, LIF, PDGF, and lipids set forth in Table 7. In a preferred embodiment, the DM comprises OSM, IL-6, PDGF and CDLM as defined in Table 5. In a further preferred embodiment, the DM is cDRFm as defined in Table 4. For example, the medium may comprise cDRFm, OSM, IL-6, PDGF and CDLM.

TABLE 7
Illustrative Combinations of OSM, IL-6, and LIF

| | Supplemented | | Supplemented with |
|---|---|---|---|
| | with IL-6-family | Supplemented | CDLM or lipids of |
| | Cytokine(s) | with PDGF | Table 6 |
| 1 | OSM | no | no |
| 2 | OSM | yes | no |
| 3 | OSM | no | yes |
| 4 | OSM | yes | yes |
| 5 | IL-6 | no | no |
| 6 | IL-6 | yes | no |
| 7 | IL-6 | no | yes |
| 8 | IL-6 | yes | yes |
| 9 | LIF | no | no |
| 10 | LIF | yes | no |
| 11 | LIF | no | yes |
| 12 | LIF | yes | yes |
| 13 | OSM, IL-6 | no | no |
| 14 | OSM, IL-6 | yes | no |
| 15 | OSM, IL-6 | no | yes |
| 16 | OSM, IL-6 | yes | yes |
| 17 | OSM, LIF | no | no |
| 18 | OSM, LIF | yes | no |
| 19 | OSM, LIF | no | yes |
| 20 | OSM, LIF | yes | yes |
| 21 | IL-6, LIF | no | no |
| 22 | IL-6, LIF | yes | no |
| 23 | IL-6, LIF | no | yes |
| 24 | IL-6, LIF | yes | yes |
| 25 | OSM, IL-6, LIF | no | no |
| 26 | OSM, IL-6, LIF | yes | no |
| 27 | OSM, IL-6, LIF | no | yes |
| 28 | OSM, IL-6, LIF | yes | yes |

D. Additional Supplements

The DM of the invention may optionally be supplemented with any number of additional supplements needed to promote the growth of cells in culture. Such supplements may include, but are not limited to, BMP family members, TGF-β family members, IGF, and insulin.

The medium of the invention can be used to seed, grow, and maintain chondrocytes capable of redifferentiation in culture without the use of serum. The stated ranges of concentrations of PDGF, lipids, OSM, IL-6, and LIF may need to be adjusted for specific cell culture conditions. Such adjustments can easily be made by a person skilled in art using routine techniques.

In some embodiments, the culture medium of the invention is not supplemented with substantially pure jagged 1 (JAG1) and/or substantially pure interleukin-13 (IL-13).

In some embodiments, the culture medium of the invention is not supplemented with any of the specific combinations of supplements set forth in U.S. Patent Application Publication Nos. US 2005/0265980 A1 (e.g., at paragraphs 59 to 68) and US 2005/0090002 A1 (e.g., at paragraphs 10 to 14), although it may be supplemented with a subset of any combination disclosed therein as long as the medium excludes at least one or more of the supplements from that combination. For example, in some embodiments, the culture medium of the invention is not supplemented with any specific one, two, three, four or more supplements selected from the group consisting of substantially pure epidermal growth factor (EGF), substantially pure stem cell factor (SCF), substantially pure insulin-like growth factor 1 (IGF-1), substantially pure brain-derived neurotrophic factor (BDNF), substantially pure erythropoietin (EPO), substantially pure FMS-related tyrosine kinase-3 (Flt-3/Flk-2) ligand, and/or a substantially pure member of the wingless-type MMTV integration site (WNT) family. In some embodiments, the medium of the invention does not contain dexamethasone.

### III. Chondrocytes and Other Suitable Cells

The methods of the invention can be used with any suitable cells. The methods are particularly suitable for ex vivo propagation of cells capable of producing cartilaginous tissue, such as chondrocytes.

Chondrocytes are cells found in various types of cartilage, e.g., hyaline cartilage, elastic cartilage, and fibrocartilage. Chondrocytes are mesenchymal cells that have a characteristic phenotype based primarily on the type of extracellular matrix they produce. Precursor cells produce type I collagen, but when they become committed to the chondrocyte lineage, they stop producing type I collagen and start synthesizing type II collagen, which constitutes a substantial portion of the extracellular matrix. In addition, committed chondrocytes produce proteoglycan aggregate, called aggrecan, which has glycosaminoglycans that are highly sulfated.

The term “chondrocyte”, as used herein, refers to a differentiated cell obtained from the cartilage, including a de-differentiated chondrocyte as grown in culture which retains the capacity to differentiate into a chondrocyte. The term “chondrocyte” refers to a chondrocyte regardless of whether it is primary or passaged, autologous, heterologous, allogeneic, xenologous, etc.

Chondrocytes used in the present invention can be isolated by any suitable method. Various starting materials and methods for chondrocyte isolation are well known in the art. Freshney, Culture of Animal Cells: A Manual of Basic Techniques, 2d ed. A. R. Liss, Inc., New York, pp. 137-168 (1987); Klagsburn, Methods Enzymol. 58:560-564 (1979); R. Tubo and L. Brown, Articular Cartilage. In: Human Cell Culture; Volume V, Koller et al. (eds.) (2001); and Kandel et al., Art. Cells, Blood Subs., and Immob. Biotech. 25(5), 565-577 (1995). By way of example, articular cartilage can be harvested from femoral condyles of human donors, and chondrocytes can be released from the cartilage by overnight digestion in 0.1% collagenase/DMEM. The released cells are expanded as primary cells in a suitable medium such as the DM of this invention or DMEM containing 10% FBS.

It may be desirable in certain circumstances to grow chondrocyte progenitor stem cells such as mesenchymal stem cells rather than cells from cartilage biopsies that are already differentiated into chondrocytes. Chondrocytes can be obtained upon differentiation of such cells into chondrocytes. Examples of tissues from which such stem cells can be isolated include synovium, placenta, umbilical cord, bone marrow, adipose, skin, muscle, periosteum, or perichondrium.

Besides chondrocytes and chondrocyte progenitor stem cells, it may be desirable in certain circumstances to utilize other cells with chondrocytic potential, such as cells of mesenchymal lineage that can be trans-differentiated into chondrocytes. Chondrocytes can be obtained by inducing differentiation of such cells into chondrocytes in vitro. Examples of such other cells with chondrocytic potential include osteoblasts, myocytes, adipocytes, fibroblasts, epithelial cells, keratinocytes, and neuronal cells.

Chondrocytes, chondrocyte progenitor cells, and other cells with chondrocytic potential may be cultured to a state that is suitable for treating a patient suffering from a cartilage defect. Such therapeutically useful chondrocytes should express articular cartilage-specific extracellular matrix components, including, but not limited to, type II collagen and proteoglycans characteristic of hyaline-like articular cartilage. Assays to determine the differentiation state of chondrocytes are known in the art and described in, e.g., R. Tubo and L. Brown, Articular Cartilage. In: Human Cell Culture; Volume V, Koller et al., eds. (2001) and the Examples.

Other cells for which the DM of the present invention may be used include any primary or passaged cells, or cells as part of cultured tissues, that are capable of growing in the DM. Examples of other cells include hepatocytes, beta cells, and islet cells.

Chondrocytes and other cells can be isolated from any mammal, including, without limitation, human, orangutan, monkey, chimpanzee, dog, cat, rat, rabbit, mouse, horse, cow, pig, elephant, etc. Cells for which the DM of the present invention may be used include any primary or passaged cells, or cells as part of cultured tissues, that are capable of growing in the DM.

### IV. Methods of Cell Culture

The cell can be cultured using any suitable cell culture methods appropriate for a particular cell type and application. Methods cell culture are well known in the art and described in, e.g., J. M. Davis, Basic Cell Culture, 2d ed. Oxford U. Press, 2002.

For example, chondrocytes can be passaged at 80-90% confluence using 0.05% trypsin-EDTA, diluted for subculture, and reseeded for second and subsequent passages to allow for further expansion. Trypsin and EDTA are both readily available from Invitrogen (Carlsbad, Calif.). Alternatively, cells may be passaged by incubation with a solution containing a chelating agent such as EDTA. The use of such chelating agents for the non-enzymatic detachment of cells is well known in the art. In a particular embodiment, cells grown in the DM of the invention are passaged using 0.1 mM to 1 mM EDTA. In a preferred embodiment, cells grown in the DM of the invention are passaged using less than 0.0025% (or 325 units/ml), preferably 0.00025% (or 32.5 units/ml), recombinant trypsin in 0.1 mM to 1 mM EDTA. At any time, cells can be collected and frozen in DMEM containing 10% DMSO and 40% HSA or in other compositions known in the art, e.g., as described in U.S. Pat. No. 6,365,405.

In some embodiments, cells can be initially cultured at low density. The term “low density” refers to seeding densities less than 20,000 cells/cm².

The methods of this invention are suitable for cells growing in cultures under various conditions including, but not limited to, monolayers, multilayers, on solid support, in suspension, and in 3D cultures.

### V. Methods of Evaluating Media

In some embodiments, a medium of the invention can be tested for the capacity to maintain cells in a differentiation-competent state, and in particular, for differentiation/redifferentiation into chondrocytes when the cells are placed in a permissive environment. Proteoglycan, aggrecan and collagen II are examples of components of the extracellular matrix normally secreted by chondrocytes in vivo and may serve as markers of chondrocyte function. The capacity of medium to maintain chondrocyte differentiation potential may be determined by agarose and/or alginate assays. The agarose assay identifies the formation of proteoglycan by cells grown in a three-dimensional agarose matrix and is described in, e.g., Benya et al., Cell 30:215-224 (1982). The alginate assay measures expression of aggrecan and collagen II genes in cells cultured in an alginate suspension and is described in, e.g., Yaeger et al., Exp. Cell. Res. 237(2):318-25 (1997); and Gagne et al., J. Orthop Res. 18(6):882-890 (2000).

### VI. Methods of Using Cells

The invention further provides cells cultured using the methods of the invention and methods of using such cells, e.g., in therapy, e.g., for treating a subject by administering to the subject such cells. For example, the methods include repair of cartilage defects (e.g., due to trauma or osteoarthritis) by administering chondrocytes (e.g., autologous chondrocytes) cultured in accordance with the methods of the invention.

### EXAMPLES

Various aspects of the invention are further described and illustrated in the Examples presented below.

### Example 1

### IL-6 Increases Cell Yield and Proliferation of Primary Human Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². 175 flasks were used for all experiments. The following media were tested:

1) DMEM+10% FBS

2) cDRF/P/L as defined in Table 8

3) cDRF/P/L as defined in Table 8, supplemented with 0.2 ng/ml IL-6

4) cDRF/P/L as defined in Table 8, supplemented with 1.0 ng/ml IL-6

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. Cell yield was greatest for cells grown in cDRF/P/L+IL-6 at all passages examined (Table 9). The growth index for cells grown in cDRF/P/L+IL-6 was roughly equal to the growth index for cells grown in DMEM+10% FBS and exceeded that of cells grown in cDRF/P/L alone (Table 10). These results indicate that cDRF/P/L supplemented with IL-6 is an effective replacement for serum-containing media.

TABLE 8
Composition of cDRF/P/L

| | 1x Liquid | |
|---|---|---|
| DRF (Table 2) | 99% | |
| ITS-X supplement (insulin, | 1% | |
| transferrin, selenium, | | |
| ethanolamine) | | |
| Supplements: | | |
| Linoleic Acid | 5 | μg/ml |
| Gentamycin | 50 | μg/ml |
| Hydrocortisone | 40 | ng/ml |
| Fibronectin | 1 | μg/ml |
| Basic Fibroblast Growth Factor | 10 | ng/ml |
| (bFGF) | | |
| Human Serum Albumin | 1 | mg/ml |
| Chemically defined lipid mixture | 5 | μl/ml |
| (CDLM) | | |
| Platelet derived growth factor | 10 | ng/ml |
| (PDGF) | | |

TABLE 9

| | Cell yield per T75, ×10⁵ | | |
|---|---|---|---|
| Medium | Passage #1 | Passage #2 | Passage #3 |
| DMEM + 10% FBS | 7.1 | 22.1 | 35.7 |
| cDRF/P/L | 13.7 | 29.1 | 26.0 |
| cDRF/P/L + 0.2 ng/ml IL-6 | 20.6 | 29.1 | 119.0 |
| cDRF/P/L + 1 ng/ml IL-6 | 20.3 | 50.1 | 118.0 |

TABLE 10

| | Growth Index (population doubling/day) | | |
|---|---|---|---|
| Medium | Passage #1 | Passage #2 | Passage #3 |
| DMEM + 10% FBS | 0.23 | 0.41 | 0.67 |
| cDRF/P/L | 0.19 | 0.27 | 0.28 |
| cDRF/P/L + 0.2 ng/ml IL-6 | 0.22 | 0.42 | 0.71 |
| cDRF/P/L + 1 ng/ml IL-6 | 0.22 | 0.54 | 0.71 |

### Example 2

### OSM Increases Cell Yield and Proliferation of Primary Human Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRF/P/L as defined in Table 8

3) cDRF/P/L as defined in Table 8, supplemented with 0.1 ng/ml OSM

4) cDRF/P/L as defined in Table 8, supplemented with 0.5 ng/ml OSM

5) cDRF/P/L as defined in Table 8, supplemented with 1.0 ng/ml OSM

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. The data in Table 11 indicates that cell yield was greatest for cells grown in cDRF/P/L+OSM at all passages examined. The growth index for cells in cDRF/P/L+OSM was roughly equal to the growth index for cells in DMEM+10% FBS and exceeded that of cells in cDRF/P/L alone (Table 12). These results indicate that cDRF/P/L supplemented with OSM is an effective replacement for serum-containing media.

TABLE 11

| | Cell yield per T75, ×10⁵ | | |
|---|---|---|---|
| Medium | Passage #1 | Passage #2 | Passage #3 |
| DMEM + 10% FBS | 12.7 | 25.6 | 23.4 |
| cDRF/P/L | 17.7 | 22.4 | 13.8 |
| cDRF/P/L + 0.1 ng/ml OSM | 37.5 | 26.4 | 48.6 |
| cDRF/P/L + 0.5 ng/ml OSM | 25.6 | 62.2 | 41.6 |
| cDRF/P/L + 1 ng/ml OSM | 22.0 | 45.8 | 41.2 |

TABLE 12

| | Growth Index (population doubling/day) | | |
|---|---|---|---|
| Medium | Passage #1 | Passage #2 | Passage #3 |
| DMEM + 10% FBS | 0.37 | 0.5 | 0.42 |
| cDRF/P/L | 0.24 | 0.32 | 0.19 |
| cDRF/P/L + 0.1 ng/ml OSM | 0.31 | 0.47 | 0.37 |
| cDRF/P/L + 0.5 ng/ml OSM | 0.33 | 0.58 | 0.50 |
| cDRF/P/L + 1 ng/ml OSM | 0.37 | 0.52 | 0.43 |

### Example 3

### LIF Increases Cell Yield and Proliferation of Primary Human Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRF/P/L as defined in Table 8

3) cDRF/P/L as defined in Table 8, supplemented with 0.1 ng/ml LIF

4) cDRF/P/L as defined in Table 8, supplemented with 0.5 ng/ml LIF

5) cDRF/P/L as defined in Table 8, supplemented with 2.0 ng/ml LIF

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. The data in Table 13 indicates that after the first passage, cell yield was greatest for cells grown in cDRF/P/L+LIF. The growth index for cells in cDRF/P/L+LIF was greater than the growth index for cells in cDRF/P/L alone after the second passage (Table 14). These results indicate that cDRF/P/L supplemented with LIF is an effective replacement for serum-containing media.

TABLE 13

| | Cell yield per T75, ×10⁵ | | |
|---|---|---|---|
| Medium | Passage #1 | Passage #2 | Passage #3 |
| DMEM + 10% FBS | 16.1 | 30.2 | 20.8 |
| cDRF/P/L | 18.5 | 34.4 | 7.8 |
| cDRF/P/L + 0.1 ng/ml LIF | 18.5 | 45.4 | 49.6 |
| cDRF/P/L + 0.5 ng/ml LIF | 18.7 | 46.6 | 44.6 |
| cDRF/P/L + 2 ng/ml LIF | 15.0 | 51.6 | 43.6 |

TABLE 14

| | Growth Index (population doubling/day) | | |
|---|---|---|---|
| Medium | Passage #1 | Passage #2 | Passage #3 |
| DMEM + 10% FBS | 0.44 | 0.54 | 0.40 |
| cDRF/P/L | 0.20 | 0.20 | 0.15 |
| cDRF/P/L + 0.1 ng/ml LIF | 0.20 | 0.40 | 0.27 |
| cDRF/P/L + 0.5 ng/ml LIF | 0.20 | 0.40 | 0.30 |
| cDRF/P/L + 2 ng/ml LIF | 0.19 | 0.54 | 0.25 |

### Example 4

### IL-6 and OSM Together Increase Cell Yield of Primary Human Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRF/P/L as defined in Table 8

3) cDRF/P/L as defined in Table 8, supplemented with 1.0 ng/ml IL-6

4) cDRF/P/L as defined in Table 8, supplemented with 0.5 ng/ml OSM

5) cDRF/P/L as defined in Table 8, supplemented with 1.0 ng/ml IL-6+0.5 ng/ml OSM

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. The data in Table 15 indicates that cell yield was greatest for cells grown in cDRF/P/L+1L-6, cDRF/P/L+OSM, or cDRF/P/L+IL-6+OSM. These results indicate that cDRF/P/L supplemented with IL-6 and OSM is an effective replacement for serum-containing media.

TABLE 15

| | Cell yield per T75, ×10⁵ | | |
|---|---|---|---|
| Medium | Passage #1 | Passage #2 | Passage #3 |
| DMEM + 10% FBS | 9.8 | 14.0 | 9.0 |
| cDRF/P/L | 14.0 | 30.3 | 32.4 |
| cDRF/P/L + 1.0 ng/ml IL-6 | 24.1 | 29.6 | 46.5 |
| cDRF/P/L + 0.5 ng/ml OSM | 19.4 | 68.0 | 30.5 |
| cDRF/P/L + 1 ng/ml IL-6 + | 16.9 | 77.3 | 91.7 |
| 0.5 ng/ml OSM | | | |

### Example 5

### JAG-1 Inhibits Growth of Chondrocytes in Serum-Free Medium

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRF/P/L as defined in Table 8

3) cDRF/P/L as defined in Table 8, supplemented with 2.0 μg/ml JAG-1

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. The data in Table 16 indicates that the cell yield in cDRF/P/L was roughly 2-fold greater than the yield in DMEM+10% FBS. However, when JAG-1 was added to cDRF/P/L, the cell yield decreased approximately 16-fold compared to cDRF/P/L alone. Likewise, the growth index for cells grown in the presence of JAG-1 was only 0.004, compared to 0.24 for cells in cDRF/P/L without JAG-1. These results indicate that, under the conditions tested, cDRF/P/L supplemented with JAG-1 is not an effective replacement for serum-containing media.

TABLE 16

| | Cell Yield | Growth Index |
|---|---|---|
| Medium | (per T75, ×10⁵) | (Pop. Doubling/Day) |
| DMEM + 10% FBS | 10.90 | 0.23 |
| cDRF/P/L | 21.70 | 0.24 |
| cDRF/P/L + 2 μg/ml JAG-1 | 1.32 | 0.004 |

### Example 6

### IL-13 Inhibits Growth of Chondrocytes in Serum-Free Medium

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRF/P/L as defined in Table 8

3) cDRF/P/L as defined in Table 8, supplemented with 3.0 ng/ml IL-13

4) cDRF/P/L as defined in Table 8, supplemented with 10.0 ng/ml IL-13

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free media were rinsed with PBS, harvested by exposure to 0.00025% Trypzean™ (0.1× recombinant trypsin; Sigma-Aldrich, St. Louis, Mo.) in 0.5 mM EDTA, counted and reseeded. Cell yield was determined and population doublings calculated at the end of each passage. The data in Table 17 indicates that the cell yield in cDRF/P/L was roughly 2-fold greater than the yield in DMEM+10% FBS. However, when IL-13 was added to cDRF/P/L, the cell yield decreased in a dose-dependent manner. IL-13 concentrations of 3 ng/ml and 10 ng/ml decreased the cell yield by 32% and 46%, respectively, compared to cDRF/P/L alone. In addition, the time in culture to required to achieve 50% to 80% confluence was increased in the presence of IL-13, resulting in a growth index of only 0.07 or 0.06. These results indicate that, under the conditions tested, cDRF/P/L supplemented with IL-13 is not an effective replacement for serum-containing media.

TABLE 17

| | Cell Yield | Growth Index |
|---|---|---|
| Medium | (per T75, ×10⁵) | (Pop. Doubling/Day) |
| DMEM + 10% FBS | 6.84 | 0.40 |
| cDRF/P/L | 14.90 | 0.21 |
| cDRF/P/L + 3 ng/ml IL-13 | 10.20 | 0.07 |
| cDRF/P/L + 10 ng/ml IL-13 | 7.98 | 0.06 |

### Example 7

### The E93 Medium Increases Cell Yield and Proliferation of Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². T75 flasks were used for all experiments. The following media were tested:

1) DMEM+10% FBS

2) cDRFm as defined in Table 4, supplemented with 5 μl/ml CDLM as defined in Table 5, 10 ng/ml PDGF, 1 ng/ml IL-6 and 0.5 ng/ml OSM (referred herein as “E93”).

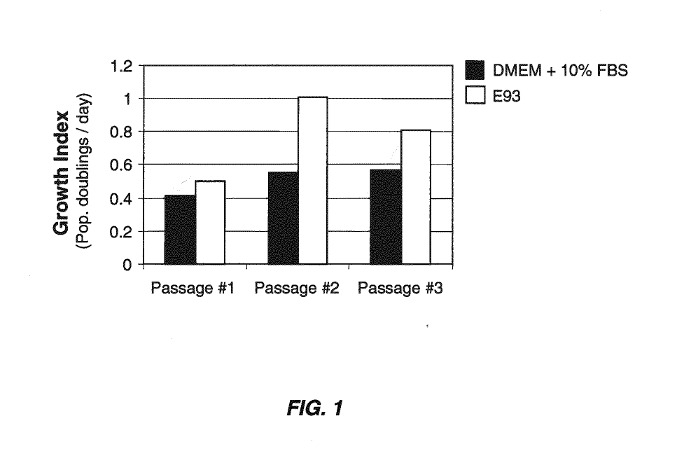

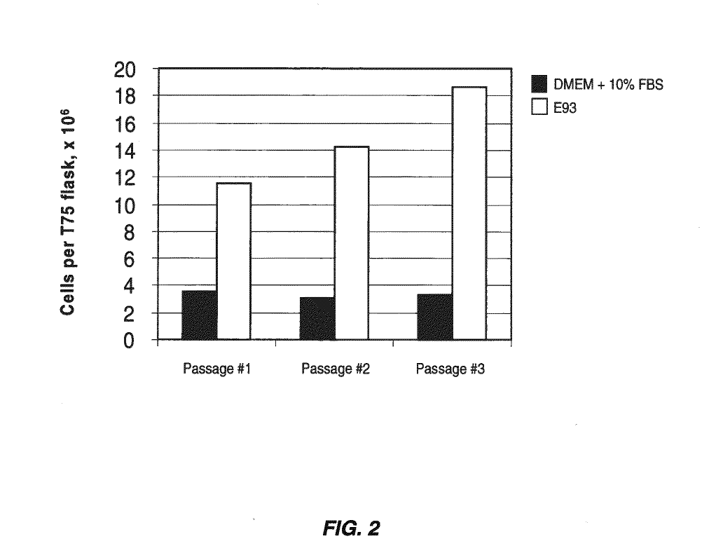

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in the E93 medium were rinsed with PBS, harvested by exposure to 0.00025% Trypzean™ in 0.5 mM EDTA, counted and reseeded. Cell yield was determined and population doublings calculated at the end of each passage. The growth index for cells in E93 was equal to or greater than the growth index for cells in DMEM+10% FBS (Table 18, FIG. 1). The cell yield for cells grown in E93 was greatly increased compared to cells grown in DMEM+10% FBS (Table 19, FIG. 2). These results indicate that cDRFm supplemented with CDLM, PDGF, IL-6 and OSM is an effective replacement for serum-containing media.

TABLE 18

| | Growth Index (population doubling/day) | | |
|---|---|---|---|
| Medium | Passage #1 | Passage #2 | Passage #3 |
| DMEM + 10% FBS | 0.41 | 0.55 | 0.57 |
| E93 | 0.50 | 1.01 | 0.81 |

TABLE 19

| | Cell yield per T75, ×10⁵ | | |
|---|---|---|---|
| Medium | Passage #1 | Passage #2 | Passage #3 |
| DMEM + 10% FBS | 35.9 | 30.8 | 33.5 |
| E93 | 116 | 143 | 186 |

### Example 8

### Medium Supplemented with IL-6 and OSM Maintains Re-Differentiation Capacity of Chondrocytes in Three-Dimensional Culture

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm² and cells grown in serum-free medium were plated at a density of 5,000 cells per cm². 175 flasks were used for all experiments. The following media were used:

1) DMEM+10% FBS

2) Serum-free E93 medium as described in Example 7

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in serum-free medium were rinsed with PBS, harvested by exposure to 0.00025% Trypzean™ in 0.5 mM EDTA, counted and reseeded.

After the third passage, cells were tested for the ability to redifferentiate as measured by colony formation and the production of proteoglycan in agarose (Benya et al., Cell 30:215-224 (1982)). Fifty thousand cells were resuspended in 2% agarose and plated in 60 mm dishes. Cells in agarose were cultured in DMEM+10% FBS at 37° C., and refed 24 hours after plating, and every 2 to 3 days thereafter. After 21 days in culture, the plates were fixed with 10% formalin, rinsed, stained with 0.2% safranin, and rinsed extensively to remove background stain. The number of colonies that stained positive for proteoglycan, and were equal to or greater than 50 microns in size, was determined. Plates on which more than 6.8% of the cells formed proteoglycan-positive colonies and met the minimum size criteria were scored as “pass”. All strains were tested in triplicate. Cell strains from six biopsies were examined. As shown in Table 17, all six strains passed the agarose assay whether they were grown in serum-containing or serum-free media. These results further indicate that cDRFm supplemented with CDLM, PDGF, IL-6 and OSM is an effective replacement for serum-containing media.

TABLE 20

| Strain No. | DMEM + 10% FBS | E93 |
|---|---|---|
| 1 | 21.5% | 14.8% |
| 2 | 13.5% | 12.3% |
| 3 | 20.8% | 11.8% |
| 4 | 36.2% | 31.3% |
| 5 | 16.6% | 17.3% |
| 6 | 15.0% | 28.4% |

### Example 9

### Mean Cell Yield for Ten Strains of Chondrocytes is Greater in Medium Supplemented with IL6 and OSM than in DMEM supplemented with serum

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm². Cells grown in serum-free medium were plated at either 5,000 cells per cm² (E93 high) or 3,000 cells per cm² (E93 low). T75 flasks were used for all experiments. The following media were tested:

1) DMEM+10% FBS;

2) E93 media, as described in Example 7.

Cells were passaged upon reaching 50% to 80% confluence. Cells grown in DMEM supplemented with 10% FBS were rinsed with PBS, harvested by exposure to 325 units/ml trypsin in EDTA, counted, and reseeded. Cells grown in E93 media were rinsed with PBS, harvested by exposure to 0.00025% Trypzean™ (0.1× recombinant trypsin; Sigma-Aldrich, St. Louis, Mo.) in 0.5 mM EDTA, counted and reseeded. A total of ten biopsies were processed to generate ten different strains. Cell yield per flask was determined at the end of each passage and normalized to the P1 yield of cells in DMEM supplemented with 10% FBS. The mean cell yield for cells grown in E93 at the high or low plating density was greatly increased compared to cells grown in DMEM supplemented with 10% FBS (Table 21). These results indicate that E93 is an effective replacement for serum-containing media.

TABLE 21

| | Normalized yield per T-75 flask | | |
|---|---|---|---|
| | (mean of 10 strains) | | |
| | P1 in DMEM + 10% FBS = 100 | | |
| Medium | Passage #1 | Passage #2 | Passage #3 |
| DMEM + 10% FBS | 100 | 68 | 86 |
| E93 low | 350 | 202 | 180 |
| E93 high | 334 | 314 | 195 |

### Example 10

### Medium Supplemented with IL6 and OSM Maintains Capacity of Chondrocytes to Re-Express Type 2 Collagen and Aggrecan in Alginate Suspension Culture

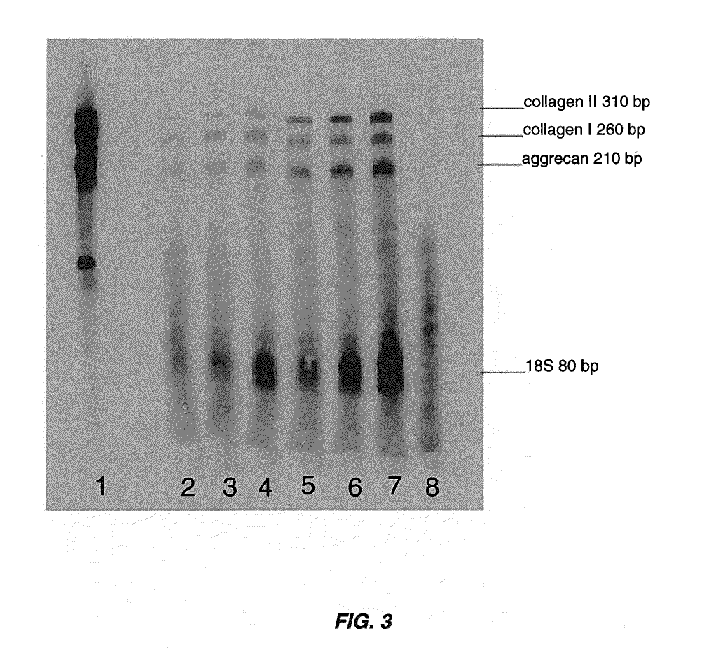

Primary human chondrocytes were prepared as describe in Example 9. Cells grown in DMEM+10% FBS or E93 were harvested in third passage for alginate culture. Alginate cultures were set up by seeding 1×10⁶ cells into a 1.2% alginate solution. Alginate cultures were fed every 3-5 days with EGHIC (DMEM, 20 ng/mL rhIGF-1, 25 μg/mL ascorbic acid, and 1 mM sodium pyruvate). After 21 days of culture, the chondrocytes were extracted from the alginate beads and mRNA for type I collagen, type II collagen and aggrecan were detected using a ribonuclease protection assay (RPA). In this assay, type II collagen is detected as a 310 base pair (bp) band on a gel, type I collagen is a 260 bp band, and aggrecan is a 210 bp band. FIG. 3 shows that increasing amounts of cell lysate from cells grown in E93 (lanes 2, 3 and 4) or DMEM supplemented with 10% serum (lanes 5, 6 and 7) contain mRNA for type II collagen and aggrecan. This indicates that human chondrocytes grown in E93 media are capable of re-expression these important cartilage markers.

### Example 11

### Karyotype and Senescence of Chondrocytes Grown in Medium Supplemented with IL6 and OSM

It may be important that cells maintain a normal karyotype and enter senescence during culture, for example, if cells are used for human therapy. Chondrocytes grown in E93 displayed a normal karyotype through at least ten passage and senesced after approximately thirty population doublings.

### Example 12

### Low Levels of Cytokines Stimulate Growth of Chondrocytes

Primary human chondrocytes were isolated from biopsies of articular cartilage by mincing of the sample followed by enzymatic digestion with 0.25% protease type XIV (Streptomyces griseus) for one hour and then 0.1% collagenase overnight at 37° C. Cells were recovered by centrifugation for five minutes at 1,000×g and resuspended in the appropriate test medium. Cells grown in DMEM+10% FBS were plated at a density of 3,000 cells per cm². Cells grown in serum-free medium were plated at either 5,000 cells per cm². T75 flasks were used for all experiments. The following media were tested:

1) DMEM+10% FBS

2) E93 media, as described in example 7

3) E93 media with 0.5 ng/ml IL-6 and 0.25 ng/ml OSM

4) E93 media with 0.1 ng/nl IL6 and 0.05 ng/ml OSM

Cells were passaged upon reaching 50% to 80% confluence. Cells grown in DMEM+10% FBS were rinsed with PBS, harvested by exposure to 325 units/ml trypsin in EDTA, counted, and reseeded. Cells grown in E93 media were rinsed with PBS, harvested by exposure to 0.00025% Trypzean™ (0.1× recombinant trypsin; Sigma-Aldrich, St. Louis, Mo.) in 0.5 mM EDTA, counted and reseeded. The growth rate, expressed as population doublings per day, was calculated at the end of each passage (Table 22). These results indicate that low levels of IL-6 and OSM in E93 support growth of primary human chondrocytes.

TABLE 22

| | Population Doublings per Day | | |
|---|---|---|---|
| | Passage | Passage | Passage |
| Medium | #1 | #2 | #3 |
| E93 | 0.40 | 0.72 | 0.63 |
| E93 w/ 0.5 ng/ml IL-6, 0.25 ng/ml OSM | 0.40 | 0.82 | 0.56 |
| E93 w/ 0.1 ng/ml IL-6, 0.05 ng/ml OSM | 0.34 | 0.72 | 0.42 |

All references cited within the specification are incorporated by reference in their entirety.

1. A culture medium comprising a basal medium supplemented with substantially pure oncostatin M (OSM).

2. The culture medium of claim 1, wherein the basal medium is additionally supplemented with substantially pure interleukin-6 (IL-6).

3. The culture medium of claim 1, wherein the basal medium is additionally supplemented with substantially pure leukemia inhibitory factor (LIF).

4. The culture medium of claim 1, wherein the basal medium is additionally supplemented with substantially pure IL-6 and substantially pure LIF.

5. The culture medium of claim 1, wherein the basal medium is cDRF.

6. The culture medium of claim 1, wherein the basal medium is additionally supplemented with substantially pure platelet-derived growth factor (PDGF).

7. The culture medium of claim 1, wherein the basal medium is additionally supplemented with one or more lipids selected from the group consisting of stearic acid, myristic acid, oleic acid, linoleic acid, palmitic acid, palmitoleic acid, arachidonic acid, linolenic acid, cholesterol, and alpha-tocopherol acetate.

8. The culture medium of claim 1, wherein the basal medium is cDRF and is additionally supplemented with PDGF and a chemically defined lipid mixture (CDLM).

9. The culture medium of claim 1, wherein the basal medium is cDRFm and is additionally supplemented with PDGF and CDLM.

10. The culture medium of claim 2, wherein the basal medium is cDRFm and is additionally supplemented with PDGF and CDLM.

11. The culture medium of claim 1, wherein the culture medium is not supplemented with substantially pure jagged 1 (JAG1) and/or substantially pure interleukin-13 (IL-13).

12. The culture medium of claim 1, wherein OSM is present at a concentration from 0.01 ng/ml to 10 ng/ml in the culture medium.

13. The culture medium of claim 2, wherein each one of OSM and IL-6 is present at a concentration from 0.01 ng/ml to 10 ng/ml in the culture medium.

14. The culture medium of claim 3, wherein each one of OSM and LIF is present at a concentration from 0.01 ng/ml to 10 ng/ml in the culture medium.

15. The culture medium of claim 4, wherein each one of OSM, IL-6, and LIF is present at a concentration from 0.01 ng/ml to 10 ng/ml in the culture medium.

16. The culture medium of claim 1, wherein the culture medium is serum-free.

17. The culture medium of claim 1, wherein the culture medium further comprises serum.

18. A culture medium comprising:
(a) cDRFm;
(b) 0.1-100 ng/ml PDGF;
(c) 0.05-5% CDLM;
(d) 0.01-10 ng/ml OSM; and
(e) 0.01-10 ng/ml IL-6.

19. A method of culturing cells, comprising the step of incubating the cell with a culture medium comprising a basal medium supplemented with substantially pure OSM.

20. The method of claim 19, wherein the basal medium is additionally supplemented with substantially pure IL-6.

21. The method of claim 19, wherein the basal medium is additionally supplemented with substantially pure LIF.

22. The method of claim 19, wherein the basal medium is additionally supplemented with substantially pure IL-6 and substantially pure LIF.

23. The method of claim 19, wherein the basal medium is cDRF.

24. The method of claim 19, wherein the basal medium is additionally supplemented with substantially pure PDGF.

25. The method of claim 19, wherein the basal medium is additionally supplemented with one or more lipids selected from the group consisting of stearic acid, myristic acid, oleic acid, linoleic acid, palmitic acid, palmitoleic acid, arachidonic acid, linolenic acid, cholesterol, and alpha-tocopherol acetate.

26. The method of claim 19, wherein the basal medium is cDRF and is additionally supplemented with PDGF and CDLM.

27. The method of claim 19, wherein the basal medium is cDRFm and is additionally supplemented with PDGF and CDLM.

28. The method of claim 20, wherein the basal medium is cDRFm and is additionally supplemented with PDGF and CDLM.

29. The method of claim 19, wherein the culture medium is not supplemented with substantially pure JAG1 and/or substantially pure IL-13.

30. The method of claim 19, wherein OSM is present at a concentration from 0.01 ng/ml to 10 ng/ml in the culture medium.

31. The method of claim 20, wherein each one of OSM and IL-6 is present at a concentration from 0.01 ng/ml to 10 ng/ml in the culture medium.

32. The method of claim 21, wherein each one of OSM and LIF is present at a concentration from 0.01 ng/ml to 10 ng/ml in the culture medium.

33. The method of claim 22, wherein each one of OSM, IL-6, and LIF is present at a concentration from 0.01 ng/ml to 10 ng/ml in the culture medium.

34. The method of claim 19, wherein the culture medium is serum-free.

35. The method of claim 19, wherein the culture medium further comprises serum.

36. The method of claim 19, wherein the cells are chondrocytes.

37. The method of claim 36, wherein chondrocytes are de-differentiated.

38. The method of claim 36, wherein the chondrocytes are derived from mesenchymal stem cells.

39. The method of claim 36, wherein the chondrocytes are human chondrocytes.

40. The method of claim 36, wherein the chondrocytes are human articular chondrocytes.

41. The method of claim 36, wherein the chondrocytes are primary.

42. The method of claim 19, further comprising the step of passaging the cells.

43. The method of claim 42, wherein the cell is passaged by incubating the cells with a solution comprising a chelating agent.

44. The method of claim 43, wherein the chelating agent is EDTA.

45. The method of claim 44, wherein EDTA is present in the solution at a concentration from 0.1 mM to 1 mM.

46. The method of claim 42, wherein the cells are passaged by incubating the cells with a solution containing less than 325 units/ml trypsin.

47. The method of claim 46, wherein the solution contains from 0.1 mM to 1 mM EDTA.

48. The method of claim 19, wherein the cells are seeded at a density less than 20,000 cells/cm².

49. A method for culturing a chondrocyte, comprising the step of incubating the chondrocyte with a culture medium comprising:
(a) cDRFm;
(b) 0.1-100 ng/ml PDGF;
(c) 0.05-5% CDLM;
(d) 0.01-10 ng/ml OSM; and
(e) 0.01-10 ng/ml IL-6.

50. A chondrocyte cultured using the culture medium of claim 1.

51. A chondrocyte cultured using the method of claim 19.

52. A chondrocyte cultured using the method of claim 49.

53. A method of treating a cartilage defect in a subject, comprising:
(a) culturing a chondrocyte using the method of claim 1; and
(b) administering the chondrocyte to the subject.

54. A method of treating a cartilage defect in a subject, comprising:
(a) culturing a chondrocyte using the method of claim 19; and
(b) administering the chondrocyte to the subject.

55. A method of treating a cartilage defect in a subject, comprising:
(a) culturing a chondrocyte using the method of claim 49; and
(b) administering the chondrocyte to the subject.

56. A composition comprising a chondrocyte and the culture medium of claim 1.

57. A composition comprising a chondrocyte and a culture medium comprising:
(a) cDRFm;
(b) 0.1-100 ng/ml PDGF;
(c) 0.05-5% CDLM;
(d) 0.01-10 ng/ml OSM; and
(e) 0.01-10 ng/ml IL-6.

58. A culture medium comprising a basal medium supplemented with:
(a) one or both of substantially pure PDGF and CDLM; and
(b) one or more of substantially pure OSM, substantially pure IL-6, and substantially pure LIF.

59. The culture medium of claim 58, wherein the basal medium is cDRF.

60. The culture medium of claim 58, wherein the basal medium is cDRFm.

61. The culture medium of claim 58, wherein the basal medium is supplemented with substantially pure PDGF and CDLM.
